(19) United States
(12) Patent Application Publication
(10) Pub. No.: US 2011/0236502 A1
(43) Pub. Date: Sep. 29, 2011

(54) Vitamin Strip (V1)

(76) Inventor: Joseph Marcel Guillory, Houston, TX (US)

(21) Appl. No.: 12/748,563

(22) Filed: Mar. 29, 2010

(51) Int. Cl. A61K 33/42; A61K 31/07; A61K 31/70; A61K 31/56; A61K 38/16; A61K 33/00; A61K 31/195; A61P 43/00

(52) U.S. Cl. 424/601; 514/725; 514/23; 514/171; 424/722; 514/565; 514/5.5; 424/641

(57) ABSTRACT

The vitamin strip is made with gel, film, or dissolvable material safe for human consumption. It can encompass all the vitamins, minerals, medicines, or meal replacement elements safe for human intake. The vitamin strip (V1) can be made as a whole or as a strip of edible dissolvable material with any vitamin(s), mineral(s), medicine(s), or meal replacement element(s) safe for human consumption on the strip of edible dissolvable material.

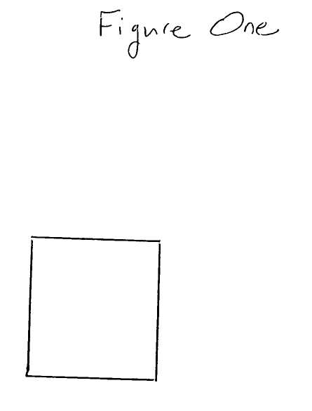

### STATEMENT OF FEDERAL FUNDING

N/A

### BACKGROUND

The problem I am overcoming with vitamin strip (V1) is the fact that pills are sickening and the vitamin strip (V1) allows people to take their vitamin(s), mineral(s), food substitute(s), or medicine(s) without water and without swallowing an entire pill. Another problem I am overcoming with the vitamin strip (V1) is the cost of manufacturing vitamin(s), mineral(s), medicine(s), and food replacement element(s). The vitamin strip (V1) is not as costly as a pill and may prove to be a lower cost than products in the vitamin, mineral, meal replacement, and medicine area.

### BRIEF DESCRIPTION OF INVENTION

The vitamin strip (V1) improves over other vitamins, minerals, medicines and meal replacements by allowing a user to securely fit the entire package of vitamin(s), mineral(s), medicine(s), or meal replacement element(s) into his or her pocket. The vitamin strip (V1) also allows an individual to take his or her vitamin(s), mineral(s), medicine(s), or food replacement element(s) without water. The vitamin strip is as stated as a strip, ribbon, or narrow piece of dissolvable material for animal consumption.

### BRIEF DESCRIPTION OF DRAWINGS

Figure One shows the vitamin strip in its complete form. The vitamin strip is a dissolvable strip of vitamin(s), mineral(s), medicine(s), or meal replacement element(s) that is designed to melt in a person's mouth.

### DETAILED DESCRIPTION OF INVENTION

The vitamin strip (V1) is a vitamin(s), mineral(s), medicine(s), or meal replacement element(s) designed to melt in a person's mouth. The vitamin strip saves time by melting in a person's mouth without the use of water. The vitamin strip (V1) is a square of a vitamin(s), mineral(s), medicine(s), or meal replacement element(s) of any type that will dissolve in a person's mouth. The vitamin strip (V1) can also be made with minerals needed to maintain a healthy body. The vitamin strip (V1) is convenient, small, healthy, and portable. The vitamin strip (V1) is around one centimeter in thickness more or less. The vitamin strip (V1) is made of dissolvable materials suitable for animal digestion. The vitamin strip (V1) saves risks of choking and can be used by adults or adolescents ages 15-103. The vitamin strip (V1) is designed to lessen risk and increase healthy animal bodies. The vitamin strip (V1) melts in an animal's mouth and may be manufactured in any color and contain any vitamin, mineral, medicine, or meal replacement element. The vitamin strip (V1) is made of film, gel, or dissolvable material that is edible by animals and is healthy for human consumption, The vitamin strip can be made of dissolvable material and liquid vitamin(s), liquid mineral(s), liquid medicine(s), or liquid meal replacement element(s). The dissolvable strip can be manufactured as one piece of dissolvable vitamin(s), medicine(s), or meal replacement element(s) and may include minerals. The vitamin strip (V1) can be manufactured for vitamin intake, mineral intake, food replacement intake, or medicine intake. The vitamin strip is not limited to the square shape but is designed as a strip of vitamin(s), mineral(s), medicine(s), or food replacement element(s) that will dissolve in an animal's mouth and is safe for human consumption,

1. The vitamin strip (V1) comprising of materials as gel, film, or dissolvable materials including vitamins A through Zink.

2. The dissolvable strip set forth in claim One further contains flavoring.

3. The dissolvable strip set forth in claim One further contains chewable material.

4. The dissolvable strip set forth in claim One further contains minerals suitable for animal consumption.

5. The dissolvable strip set forth in claim One further contains medicine suitable for animal consumption.

6. The dissolvable strip set forth in claim One further contains Fat suitable for animal consumption.

7. The dissolvable strip set forth in claim One further contains a Carbohydrate or Carbohydrates suitable for animal consumption.

8. The dissolvable strip set forth in claim One further contains a Calorie or Calories suitable for animal consumption.

9. The dissolvable strip set forth in claim One further contains Cholesterol suitable for animal consumption.

10. The dissolvable strip set forth in claim One further contains Protein suitable for animal consumption.

11. The dissolvable strip set forth in claim One further contains Sugar suitable for animal consumption.

12. The dissolvable strip set forth in claim One further contains Fiber suitable for animal consumption.

13. The dissolvable strip set forth in claim One further contains Potassium suitable for animal consumption.

14. The dissolvable strip set forth in claim One further contains Calcium suitable for animal consumption.

15. The dissolvable strip set forth in claim One further contains Magnesium suitable for animal consumption.

16. The dissolvable strip set forth in claim One further contains Acid suitable for animal consumption.

17. The dissolvable strip set forth in claim One further contains Sodium suitable for animal consumption.

18. The dissolvable strip set forth in claim One further contains Phosphorus suitable for animal consumption.

19. The dissolvable strip set forth in claim One further contains Creatine suitable for animal consumption.

20. The dissolvable strip set forth in claim One further contains coloring.
